(19) United States
(12) Patent Application Publication
(10) Pub. No.: US 2012/0218514 A1
(43) Pub. Date: Aug. 30, 2012

(54) MEDICAL DEVICE

(75) Inventor: Tsong Yun Kwong, Middlesex (GB)

(73) Assignee: MEDIVENTION LTD, Wembley Park, Middlesex (GB)

(21) Appl. No.: 13/500,818

(22) PCT Filed: Oct. 7, 2010
(86) PCT No.: PCT/GB2010/051678

§ 371 Date: May 21, 2012

(30) Foreign Application Priority Data
Oct. 7, 2009 (GB) 0917479.8
Oct. 22, 2009 (GB) 0918525.7
Dec. 1, 2009 (GB) 0920950.3

(51) Int. Cl. A61B 3/12; A61B 5/11; A61B 3/024; A61B 5/12; B32B 1/08; A61B 1/227; A61B 1/06

(52) U.S. Cl. 351/205; 600/197; 600/553; 600/188; 600/559; 428/34.1; 428/364; 428/397; 351/224

(57) ABSTRACT

The present invention relates to a device comprising a shaft having at least a first portion and a second portion that can adopt a closed position in which the first and second portions are positioned adjacent each other and an open position in which the first and second portions are positioned distant from each other, one or both of the first and second portions housing or forming part of one or more of an ophthalmoscope, an otoscope, a tendon hammer, a tuning fork and a red hat pin. Also provided are improved otoscope heads and means to protect the functional heads of medical tools when not in use.

The present invention relates to improved diagnostics tools for clinical diagnosis, particularly but not exclusively tools for neurological and ear/nose and throat examination (ENT).

During examination it is essential that a doctor has a range of instruments at his disposable to perform a neurological examination and ENT examination. However this kit is often heavy and cumbersome and as a result doctors often do not perform a full physical examination on their patients.

Even basic kit is not inexpensive and weighs around more than 1kg. Very often the tools in a hospital or general practitioner surgery environment are unavailable or non-functioning leading to clinicians spending valuable time hunting for these tools.

In particular with an ophthalmoscope and otoscope, there is often one common battery shaft with connections to an ophthalmoscope head and otoscope head. These heads are easy to damage and expensive and because of the need to have two heads, bulky and not portable. When not in use, the head is typically stored in a case. When the device is in use, the head is not protected and this is often a cause of equipment failure because the instrument can be dropped on the floor causing damage.

The present invention seeks to provide a solution to these disadvantages by providing a diagnostics tool which incorporates more than one diagnostic device. The diagnostic devices include, but are not limited to, two or more of an ophthalmoscope, which is used to look at patient's retinas and pupils, an otoscope, which is used to look at patient's ear canals, a red hat pin to assess field of vision, a tendon hammer to examine reflexes and a tuning fork, used to assess a patient's hearing or as part of the examination of the peripheral nervous system. The tool provided is preferably a single, compact tool.

The present invention also aims to provide a solution to prevent damage to the medical instrument's delicate functional head and so reduce maintenance costs to hospital and physicians' budgets.

In a first aspect the present invention provides a device comprising a shaft having at least a first portion and a second portion that can adopt a closed position in which the first and second portions are positioned adjacent each other and an open position in which the first and second portions are positioned distant from each other, one or both of the first and second portions housing or forming part of one or more of an ophthalmoscope, an otoscope, a tendon hammer, a tuning fork and a red hat pin.

In one embodiment one or both of the first and second portions house or form a handle for the tendon hammer. The handle of the tendon hammer may be telescopic. The first and second portions may be in the closed position when they form the handle for the tendon hammer.

In one embodiment the tuning fork is electronic.

The first and second portions of the shaft are preferably accurate in cross section and elongate. The first and second portions are preferably sized and shaped such that when in the closed position the resultant shaft is any suitable shape that is comfortable to hold, for example circular or ovular in cross section and elongate. The first and second portions may be of the same or different cross sections.

The first and second portions may each have an outer surface that, when in the closed position forms the outer surface of the shaft, that faces the user, and an inner surface which when in the closed position faces into the centre of the shaft. The shaft formed by the first and second portions positioned adjacent each other may be hollow along all or part of its length.

The first and second portions of the shaft each have a first end and a second end and are preferably secured together by means of a hinge and/or a pivotal connection between the second ends, or areas of the shafts close to the second ends. Alternatively the first and second portions may be releasably secured together at one or more points along their length, for example by appropriate male and female engaging parts.

One of the first and second portions may be provided with an ophthalmoscope head secured thereto or integrated therein. The ophthalmoscope head may be secured to or integrated with the inner surface of the first or second portion. The ophthalmoscope head may be secured or integrated close to the first end of the first or second portion, for example between the midpoint along the length and the first end of the first or second portion.

One of the first and second portions may be provided with an otoscope head secured thereto or integrated therein. The otoscope head may be secured to or integrated with the inner surface of the first or second portion. The otoscope head may be secured or integrated close to the first end of the first or second portion, for example between the midpoint along the length and the first end of the first or second portion.

In one embodiment an ophthalmoscope head is secured to or integrated in one of the first and second portions and an otoscope head is secured to or integrated in the other of the first and second portions.

The first and second portions may individually or together act as shafts for the ophthalmoscope and otoscope as appropriate.

One of the first and second portions of the shaft may be provided with the handle of a tendon hammer permanently secured thereto. The shaft of the tendon hammer may be permanently secured to the inner surface of the first or second portion. The portion of the shaft having the handle of the tendon hammer secured thereto may be provided with an aperture to receive and expose the head of the tendon hammer. The aperture may be provided near to the second end of the relevant portion of the shaft, for example between the mid point along the length and the second end of the portion. The handle of the tendon hammer may be telescopic to allow the head to be extended clear of the shaft and the shaft to be used as part of the handle of the hammer.

There may be provided a tuning fork as part of the tool. This can be sited within the shaft or can be an extension from the second end of one of the first and second portions. This is preferably electronic in nature.

A red hat pin may be secured to one of the first and second shaft portions. Appropriate means may be provided to releasably secure the red hat pin to the inner or outer surface of the first or second portion of the shaft.

The Ophthalmoscope

It is envisaged that the ophthalmoscope will be based on standard and well known existing commercial design.

This includes but not exclusively, a light source such as lamp or LED, a mirror in the shaft to direct light to a patient, a series of lenses in a lens track which may be based in the head. Traditionally lenses are stored in a rack in the head of the ophthalmoscope.

Our tool proposes a preferred embodiment in the shaft of the tool, running over top of the viewing aperture.

There is therefore provided an ophthalmoscope having a one or more lenses held in a lens track wherein the lens track runs over the top of the viewing aperture.

Accordingly the lens track runs in a plane parallel to or co-extensive with the longitudinal axis of the shaft.

In existing ophthalmoscopes the lens track runs around the viewing aperture in a sideways circular motion and therefore runs in a plane perpendicular to the longitudinal axis of the shaft.

The lenses are held in a track system, where there are gaps between the lens. A plurality of lenses is used of differing dioptre strength. The purpose of these is lens is correct for refractive differences between the user and the patient's eye. The lens would be positioned in such a fashion that there would be a suitable gap between lenses on the track to avoid the situation where the user would be viewing the eye with two lenses. The lenses can be controlled with an electronic or mechanical switch on the shaft of the tool that can be operated by preferably the thumb or index finger.

An innovative feature of having the lens track coming over the ophthalmoscope head rather than around the side is that it allows a narrower size head to be used, which will increase the portability of the device. It also allows the lens track to be more likely changed with the thumb which can be more comfortable.

The lens track is supported by two or more wheels, around which the track passes. There could also be a mechanism to be able to attach an add-on to the ophthalmoscope head to convert it to an otoscope.

The Otoscope

An otoscope is used to examine the ear canals of patients. Standard design reveals a device, which has a battery to power a light source such as a lamp or LED. A mirror is generally used to direct the light into ear canal however more recent otoscopes use a fibreoptic system to direct the light without the need for a reflection mirror. There is a magnifying lens one side for the doctors' eyes, and a protruding element on the other side. This element allows a disposable ear speculum to be attached and can be used as an outlet for a fibreoptic system. The lighting source can be integrated into the protruding element or may be passed through it.

Electronic Tuning Fork

Preferably, the tool of the invention will generate a plurality of acoustic tones, each at a predetermined frequency and able to generate a vibration of a predetermined frequency. These frequencies may include and are not limited to 128 Hz, 256 Hz, 512 Hz and 1024 Hz.

In addition, a speaker system can be integrated that can mimic the tone of the vibration that is necessary for conducting hearing tests.

A further function may also be provided to allow the frequencies to decay at a predetermined rate. Any resulting vibration can have mechanism so that it can be directed to a set point along the tool. The tool may include a switch or buttons to choose the frequency. Likewise a speaker may also have a decaying tone.

The tool may include a visual display to show the frequency of the vibration chosen, battery levels, on and off. The visual display may be linked with the switch system.

The visual display may include having touch screen technology.

Within the ophthalmoscope, there may be a camera to take digital images of the retina. Likewise there maybe a camera in the otoscope to take images of the inner ear. These devices may be connected to a storage device and there may be and in one embodiment there is also a means to retrieve the images from the storage device into distribute electronic media. A playback function maybe incorporated and may use the visual display.

In the preferred embodiment, there maybe a sound recorder to record sound files with an inbuilt attached microphone. The storage device as described earlier could be used to store the recordings or a separate storage device may be used. The visual display may be used to select the function of the sound recording equipment, including but not exclusively the use of touch screen technology. Otherwise there will be buttons to select the different function such as play, record, rewind and forwards.

There will/may be electrical sockets for input/output including and not exclusive to earphones, connection to a stethoscope.

It is envisaged that an electrical circuit may control functions of the tool, including but not exclusive to the tuning fork frequencies, connections to the electronic display, a power circuit.

Within the shaft, it is envisaged that storage device may be stored. The storage device may alternatively be secured to or be an integral part of one of the first and second portions.

In its preferred embodiment, the tool will contain a slot for the containment of a red hat pin, which may be locked into the tool and removed from the tool via a locking mechanism.

In its preferred embodiment, the tool includes an electric battery to energise the ophthalmoscope, otoscope and any other component within the device requiring this type of energy to operate. This battery is preferably held in the shaft of the tool, or may be secured to one of the first and second portions of the shaft. The battery may be recharged by an external means through a connection on the shaft.

Otoscope Improvement

A technical issue with current otoscope is that is not streamline, it is fixed in shape and bulky. An improvement is suggested as follows.

In a second aspect of the invention there is provided an otoscope head having a light source and magnifying lens and a protruding element for attachment of an ear speculum, wherein the protruding element is telescopic in nature and can be extended for use and retracted for storage.

In its preferred embodiment, the protruding part is made of cylindrical shafts, which can be extended out or remain in a closed position. There is a switch, which allows or causes the protruding parts to open and close. There may alternatively be a lock that allows the protruding parts to open and close. Light may be directed through the shafts. With this means, it allows the otoscope to be streamline and hence more portable.

A secondary embodiment may be developed to turn an ophthalmoscope head into otoscope head with the use of an additional detachable secondary device.

In a third aspect of the invention there is provided a device comprising one or both of a magnifying lens, provided with means to secure it to an ophthalmoscope, and a protruding element for attachment of an ear speculum, provided with means to secure it to an ophthalmoscope.

In one embodiment the device comprises both a magnifying lens and a protruding element for attachment of an ear speculum and is provided with one or more means to secure it to an ophthalmoscope. The or each means to secure the magnifying lens and the protruding element to the ophthalmoscope may be provided with means to receive the magnifying lens and the protruding element and means to secure these to the ophthalmoscope.

The protruding element may be telescopic in nature such that it can be extended for use and retracted for storage.

In this preferred embodiment, the device of the third aspect of the invention contains three sections to fit around ophthalmoscope head. In its preferred embodiment, each section is separate and attached to an adjacent section by a link, but this may not be so, other arrangements are envisaged. One end of the device of this embodiment contains a magnifying lens; the other end has grooves or cylindrical shafts to accommodate a speculum. The shafts may be connected to a switch to cause them to open out or retract or a lock to allow them to be opened out or retracted.

The device of the third aspect of the invention may also be straightened out for storage, for example to be attached to the shaft of the ophthalmoscope or the shaft of a tool according to the first aspect of the invention, or brought into a shape to accommodate the ophthalmoscope head. In one embodiment this movement between two positions is allowed for by the use of linked sections but could be achieved in any other suitable way.

In a fourth aspect of the invention there are also provided mechanisms to protect the functional head by either allowing the functional head to be retracted or rotated into the shaft or to have a protecting mechanism such as a cover to protect it from external force and damage. It is envisaged that these types of protection mechanisms can be used on individual instruments or in multi-functional tools, for example as described in the first and/or second aspects of the invention, where there may be a plurality of guards used, which may be of one type or a combination of different types depending on the design of the tool.

In an embodiment of this further aspect the present invention provides a medical tool having a head and a shaft wherein the shaft is provided with a suitable shaped, sized and positioned cavity and the head is mounted on the shaft by such means that the head can adopt an open position in which it is exposed ready for use and a closed position in which it is housed within the cavity in the shaft for protection.

The head may be secured at or close to one end of the shaft. The head may be secured to the shaft by any suitable means for example by means of a hinge or by means of a point of rotation or pivot.

Alternatively, the head may be secured within the shaft by a resilient material or device, for example a coil spring, that favours pushing the head into the open position but will allow receipt of the head within the cavity.

Means may be provided to lock the head in one or both of the open and closed positions.

In a further embodiment of this fourth aspect of the invention there is provided a medical tool having a shaft and a head, wherein the shaft is provided with a guard movably secured thereto that in an open position exposes the head for use and in a closed position covers all or part of the head for protection.

The guard may be secured to the shaft by means of one or more hinges.

Alternatively the guard may be slidingly moveable in relation to the shaft, for example the guard may be secured to the shaft by a runner and track/groove arrangement. The or each groove may be provided on the outer surface of the shaft or the inner surface of the guard and the or each corresponding runner may be provided on the inner surface of the guard or the outer surface of the shaft respectively.

Means may be provided to lock the guard in one or both of the open and closed positions.

Embodiments of the various aspects of the present invention will now be described in further detail with reference to the following figures; the embodiments described are intended only to exemplify aspects of the invention and are not intended to limit the scope of protection in any way:

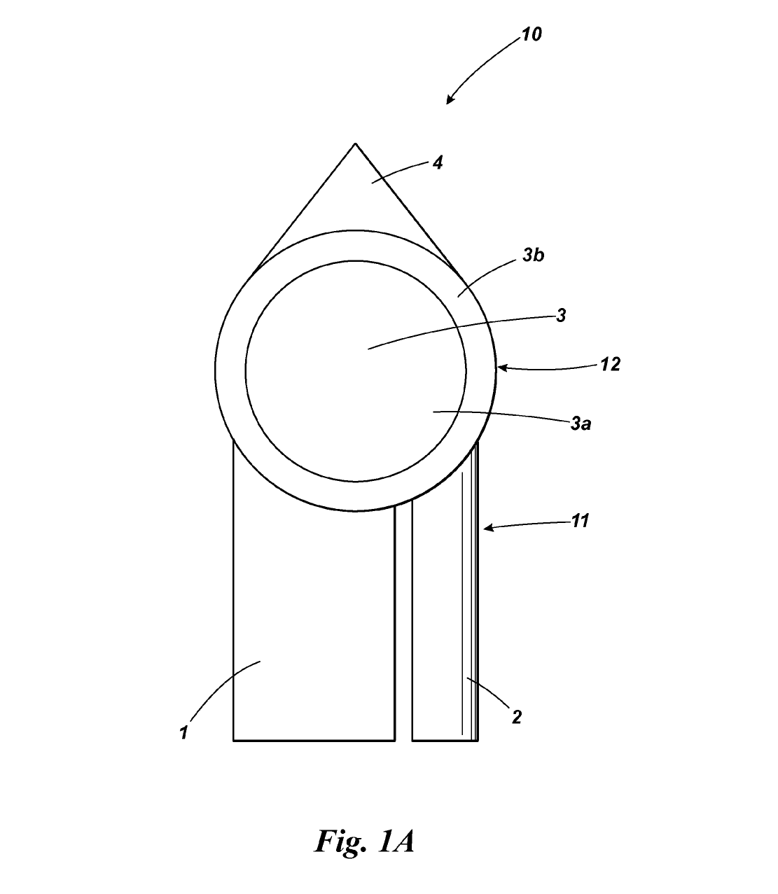

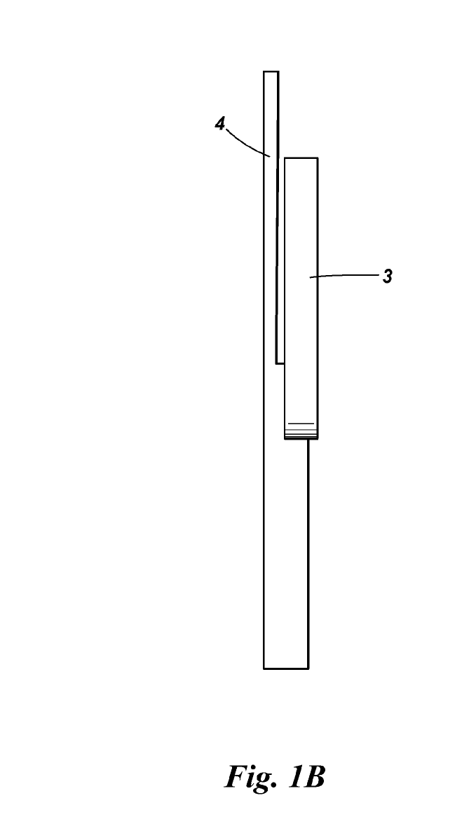

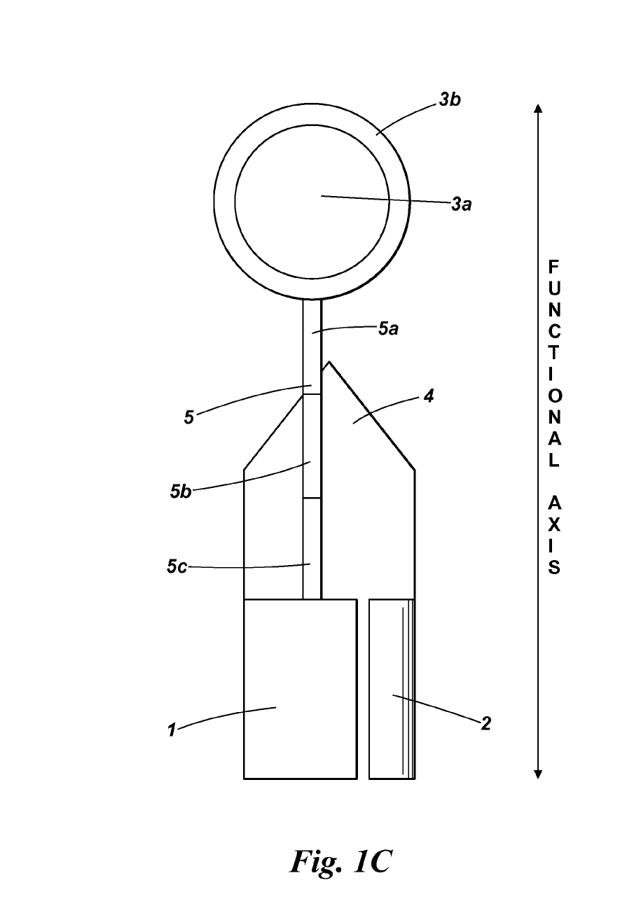

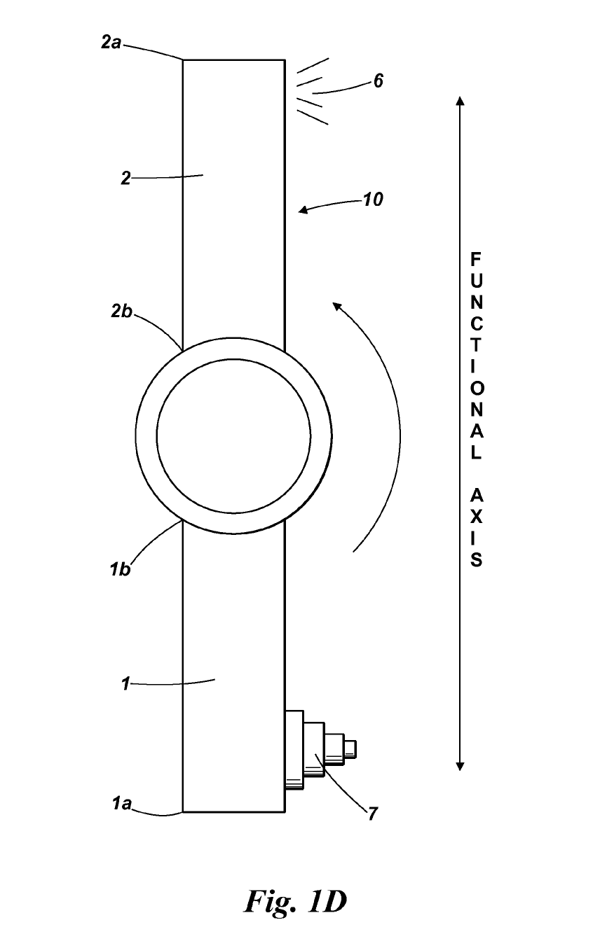

FIG. 1 shows views of the first aspect of the invention in open and closed states, in particular:

FIG. 1a shows a front view of an embodiment of a device of the first aspect of the invention in the closed state;

FIG. 1b shows a side view of the device of FIG. 1a;

FIG. 1c shows a front view of the device of FIG. 1a with the tendon hammer in the extended position;

FIG. 1d shows a front view of the device of FIG. 1a in the open state;

FIG. 1e shows a front view of an embodiment of a device of the first aspect of the invention in the closed state showing the control and connection positions;

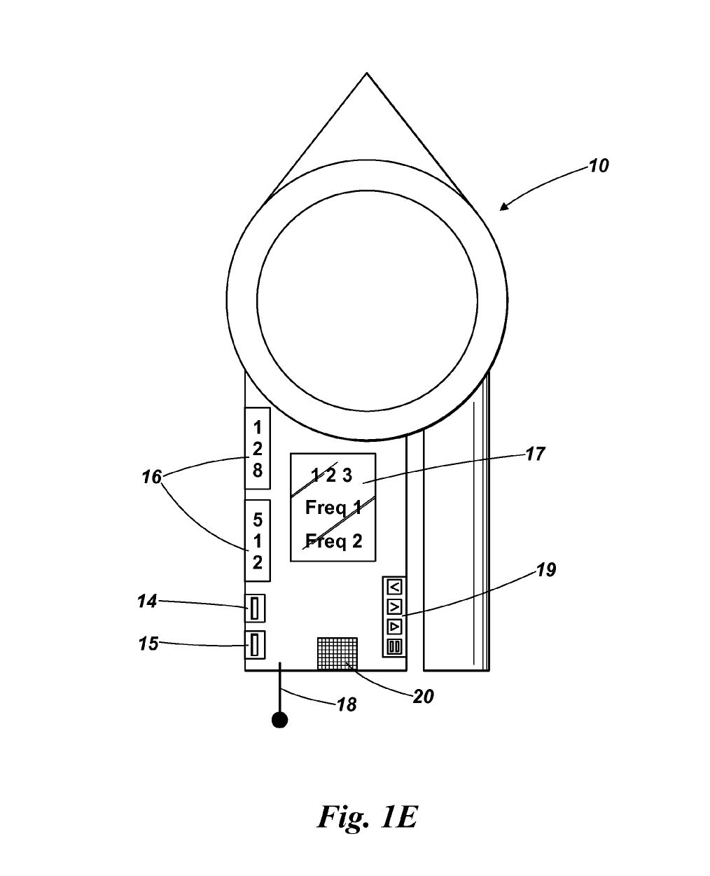

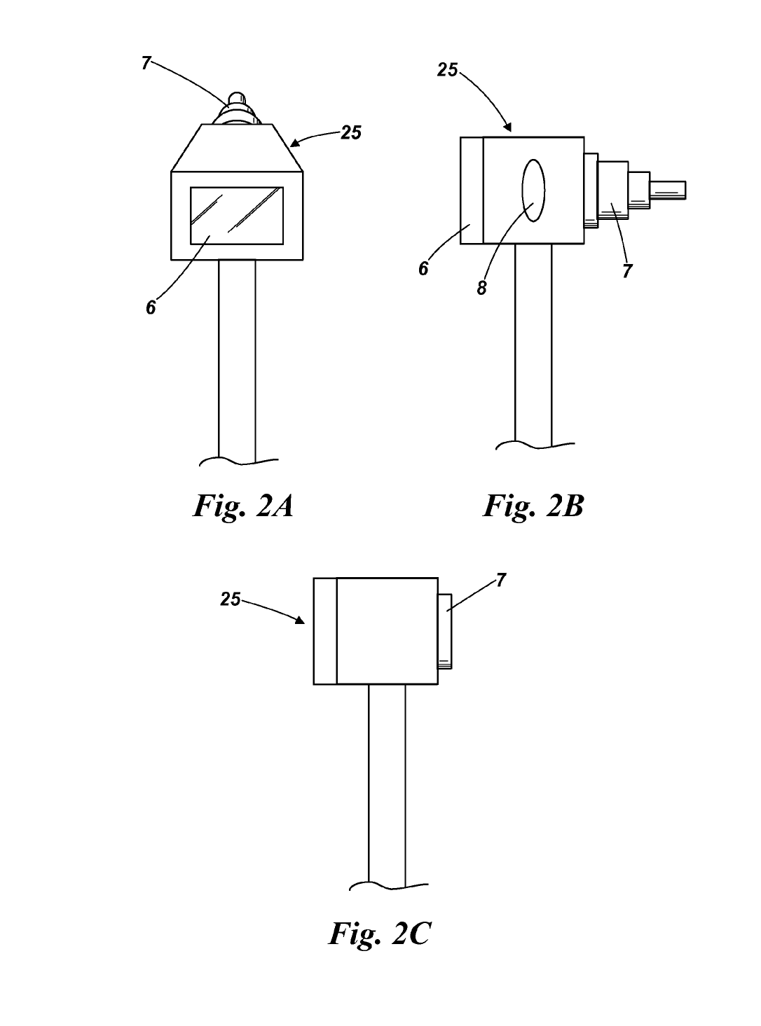

FIG. 2 show the otoscope design of the second aspect of the invention, in particular:

FIG. 2a shows a perspective view of an embodiment of the otoscope of the second aspect of the invention in the ready to use position;

FIG. 2b shows a side view of the otoscope of FIG. 2a;

FIG. 2c shows a side view of the otoscope of FIG. 2a in the retracted position for storage;

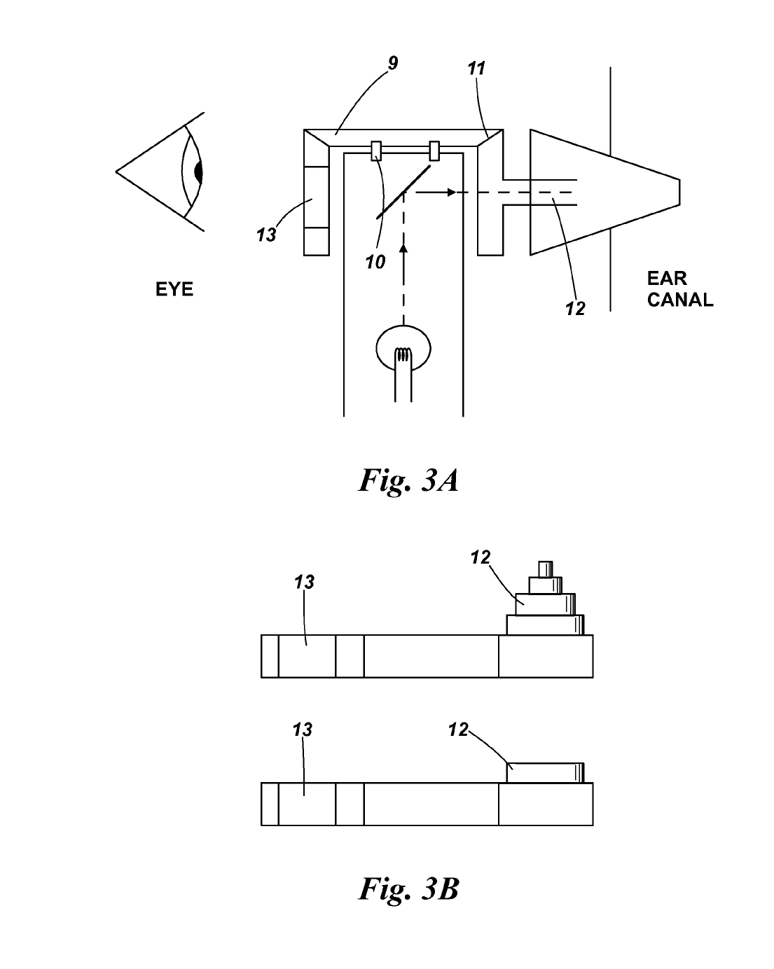

FIG. 3 show the ophthalmoscope add-on of the third aspect of the invention, in particular;

FIG. 3a shows a cross section through an ophthalmoscope and an embodiment of the add-on of the third aspect of the invention;

FIG. 3b (1) and (2) show a side view of the add-on in the ready to use and the retracted state;

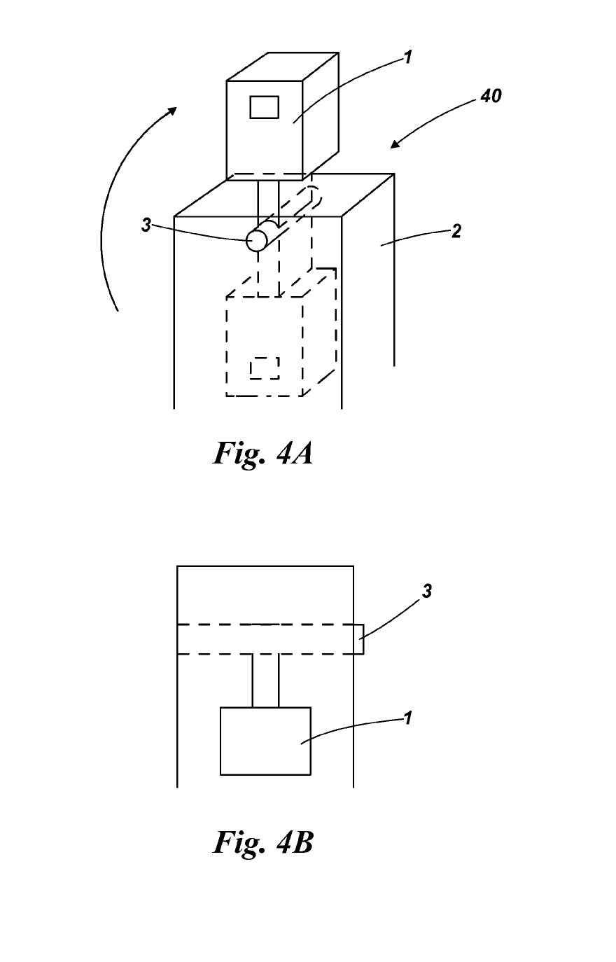

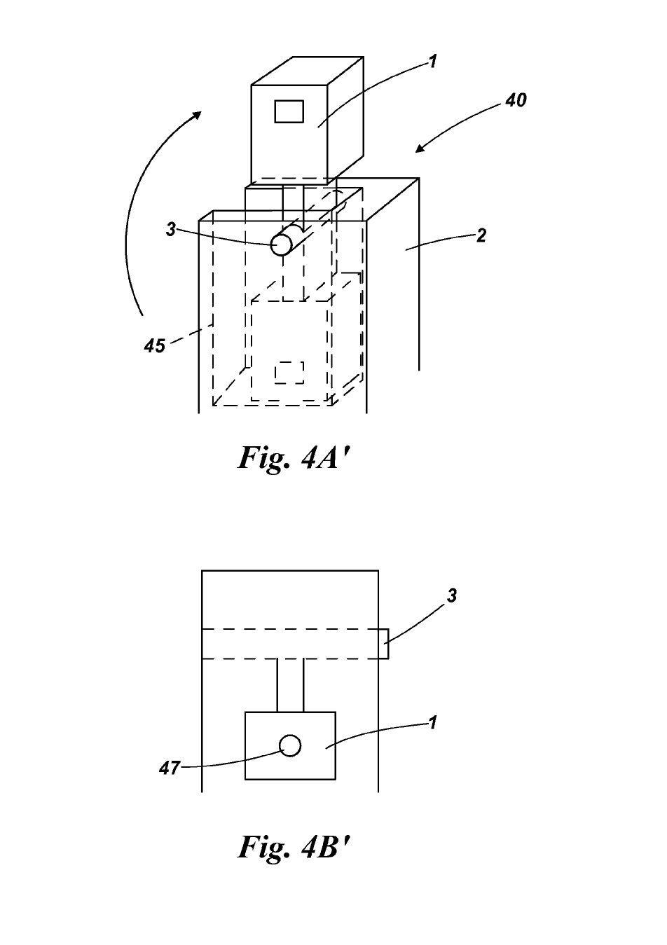

FIG. 4a/4a′ and 4b/4b′ show an embodiment of the fourth aspect of the invention in open ready to use and retracted states respectively;

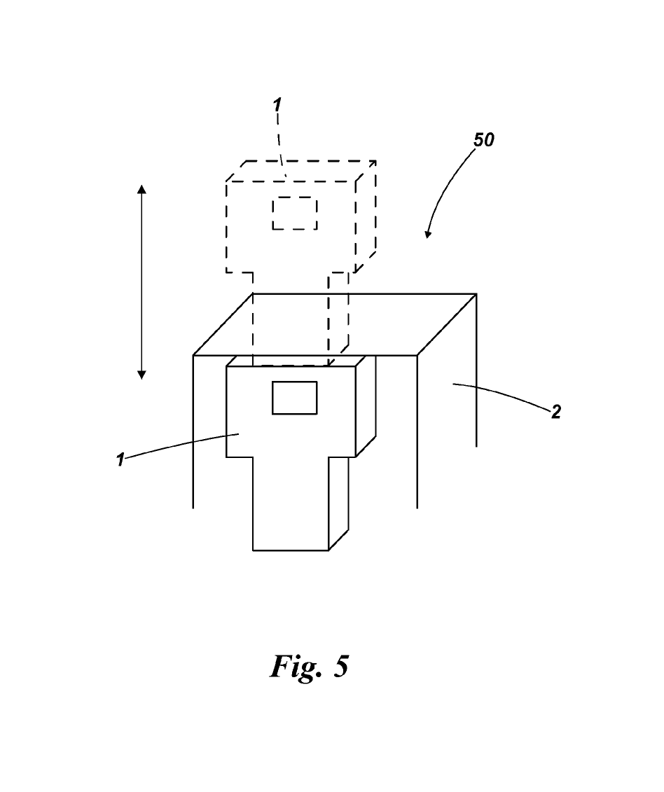

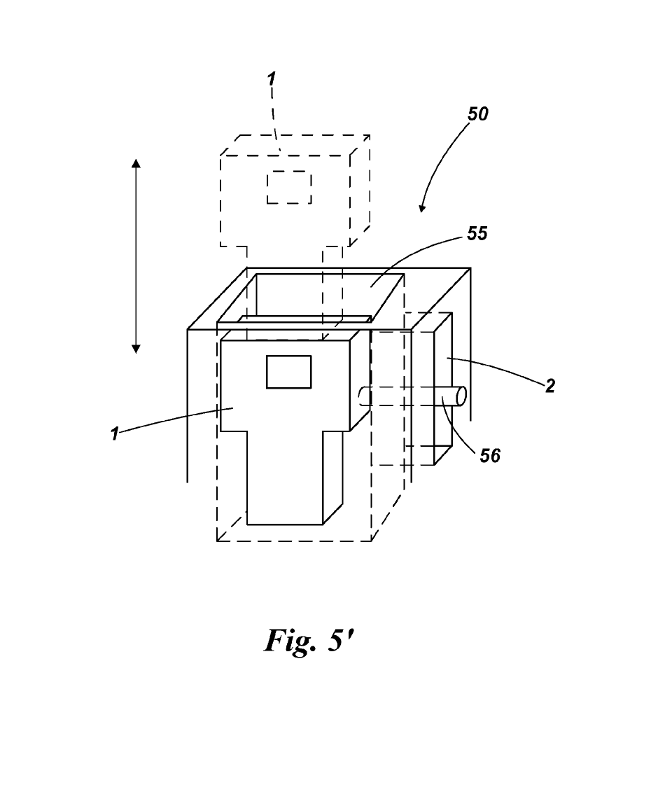

FIG. 5/5′ shows an alternative embodiment of the fourth aspect of the invention;

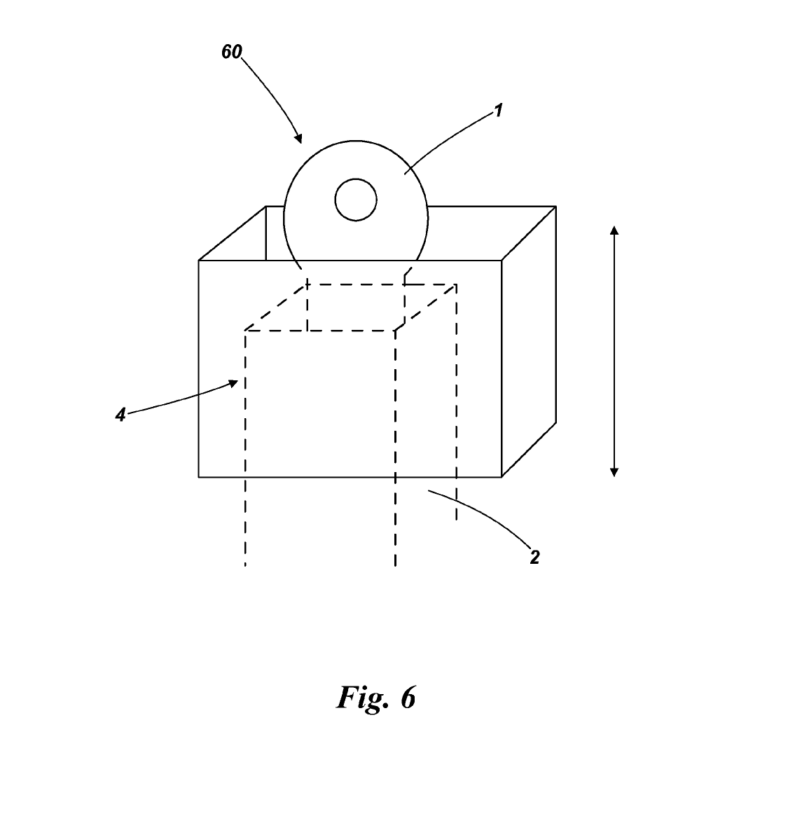

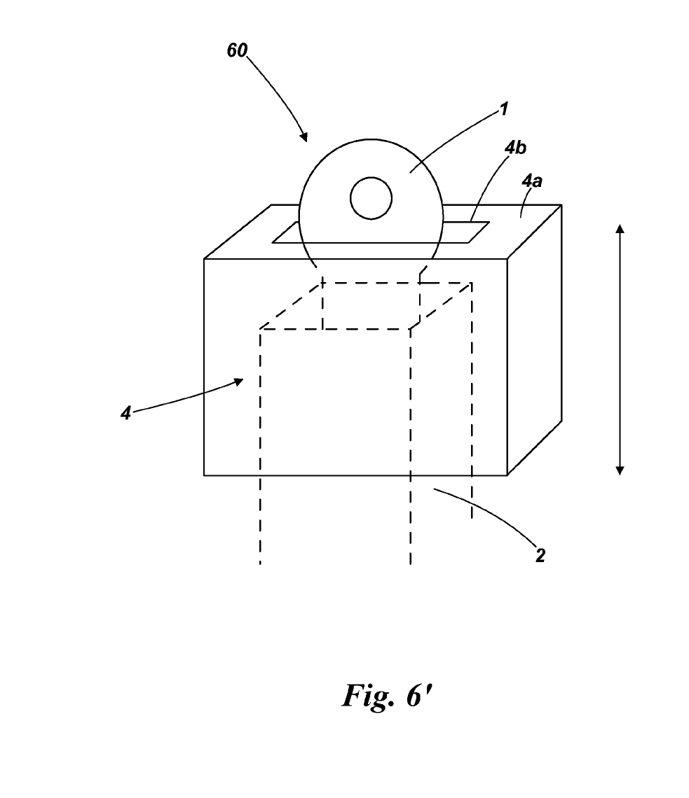

FIG. 6/6′ shows a yet further embodiment of the fourth aspect of the invention; and

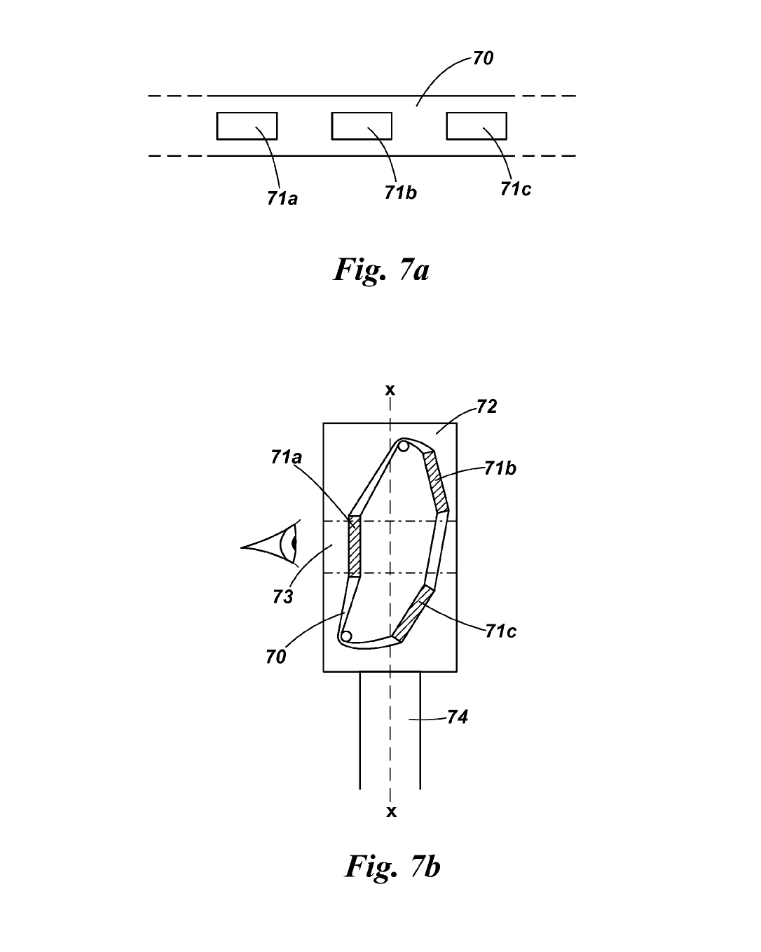

FIGS. 7a and 7b show a lens track for an ophthalmoscope in accordance with an aspect of the invention.

FIGS. 1a, 1b and 1c show a diagnostic tool 10 in accordance with an embodiment of the first aspect of the invention. The tool comprises a shaft 11 made up of a first portion 1 and a second portion 2. The first and second portions are each arcuate in cross section and elongate in length. The first and second portions each have an inner surface that faces the centre of the shaft and an outer surface that forms the outer surface of the shaft and faces the user. The first 1 and second 2 portions have a closed position, as shown in FIG. 1a where they are positioned adjacent each other to form shaft 11 which is elongate and circular in cross section. The first and second portions may differ in cross section as shown in FIG. 1a.

Also shown in FIGS. 1a, 1b and 1c is a tendon hammer 12 having a head 3 and a shaft 5. The head 3 protrudes from an aperture (not visible) in the first portion 1 of the shaft 11 or from an end of the portion 1. The head 3 is secured to the first portion 1 of the shaft 11 by means of its shaft 5, which is secured to the inner surface of the first portion 1. The shaft 5 is secured to the inner surface of the first portion 1 by, for example, an appropriate adhesive or it may be formed integrally with the first portion 1.

The tendon hammer head 3 will consist of a central disc 3a made of a metal such as stainless steel and a rubber ring 3b surrounding the central disc. The shaft of the tendon hammer is made of a number of telescopic sections 5a, 5b, 5c to allow it to be stored in the tool in the retracted position and telescopically extended out of the shaft 11 for use. The shaft of the tendon hammer 5 is made of a lightweight material such as aluminium.

In use the tendon hammer would be moved to the extended position by pulling the head 3 such that the shaft 5 telescopically extends out of the first portion 1 of the shaft. The shaft 11 then forms a handle for the tendon hammer in use. When the tendon hammer has been finished with the head 3 can be pushed towards the shaft and the shaft 5 telescopically retracts back into the shaft 11.

The tool 10 is also provided with a focal point 4 of the instrument for the vibration. This is a protrusion from the second end of one of the first and second portions of the shaft 11. The protrusion may be separate to or integral with one of the first and second portions of the shaft 11. There would be provided a battery operated vibration generator (e.g. piezoelectric device) sited at the point 4 to cause vibration of the protrusion. The tip of the tool is chosen because it can provide a precise point of vibration required for neurological vibration examination. The vibration would be isolated in the tip so that the whole tool does not vibrate when in use.

FIG. 1d shows the tool 10 with the first 1 and second 2 portions in the open position. The first and second portions each have first 1a, 2a and second 1b, 2b ends and are secured together at their second ends 1b, 2b by an appropriate hinge or pivot arrangement, for example the second ends 1b, 2b of each of the first and second portions 1, 2 have two holes provided extending through them. The holes are arranged such that in each of the first and second portions they are as circumferentially distant as possible and those of the first portion correspond with those of the second portion and a bolt can pass through the holes of both the first and second portions securing the first and second portions together and providing a pivot point. The first ends 1a, 2a of each of the first and second portions are free ends.

The first portion 1 of the shaft 11 is provided with an otoscope head 7 positioned close to the first end. The second portion 2 of the shaft 11 is provided with an ophthalmoscope head 6 positioned close to the first end 2a thereof.

To use the otoscope 7, the user would move the first and second portions into the fully open position, spaced apart by 180°, and would then hold onto the second portion of the shaft using the tendon hammer head 3 as a grip.

It is envisaged that there could be a button on the side of that arm/second portion that allows the light to be turned on and off. There may be a separate dial to vary the intensity of the tool and that circuitry would be in the shaft of the tool.

To use the ophthalmoscope 6, the user would move the first and second portions into the fully open position, spaced apart by 180°, and would then hold onto the first portion of the shaft using the tendon hammer head 3 as a grip. It is envisaged that there could be a button on the side of that arm/first portion that allows the light to be turned on and off.

FIG. 1e shows the possible external features of the tool 10. These can include a screen 17, which will have a multitude of functions including but not limited to; touch screen allowing the user to choose functions of the tool, pictures taken by the cameras on the device. There will be buttons/switches 16 to allow a selection of the frequencies for the tuning fork element but not limited to those on the diagram in terms of number. There may be input/output sockets 14, 15 for the tool including but not limited to headphones, output to hardware such as computers, an electrical charger point, USB port. The tool 10 may contain sound recording abilities especially if a stethoscope is to be incorporated in its design, and controls 19 would available for playback, recording, rewind and fast forward. There may be a microphone 20 that may be used to record sounds.

A red hat pin 18 may be stored against the inner surface of the first portion 1 and may be held in place by a suitable locking mechanism in the tool and be easily obtainable.

FIGS. 2a, 2b and 2c show an embodiment of an otoscope head 25 according to a second aspect of the invention. FIGS. 2a and 2b show the otoscope in its useable state and it can be seen that it comprises a magnifying lens 6 through which the clinician looks and a protrusion component 7 that the ear speculum sits on. The protrusion component 7 is made of telescopic parts (which are generally very small), which interlock when they are extended. This may be controlled or by a switch or button, either mechanically or electrically (8).

FIG. 2c shows this device when telescopic parts are retracted into a closed position. It can be seen that such a configuration allows the otoscope, when not in use, to be of a more streamlined shape so it is less likely to be damaged and more readily portable.

The otoscope head 25 of FIGS. 2a-2c could be used with a tool of the first aspect of the invention or with any other existing otoscope shaft arrangement.

FIGS. 3a and 3b show an embodiment of the add-on part of the third aspect of the invention, which may be used to convert an ophthalmoscope into an otoscope. In FIG. 3a, the add-on 9 is divided into three sections. No limit is placed on the number of sections needed for the device. At each join 11 it is envisaged that joint is flexible, for example hinged, but can be locked in a given position. The add-on can be held into position with a locking mechanism 10 between the ophthalmoscope head and add-on. The ear speculum is held into place with a protrusion part 12, which may be made of telescopic tubular members or cylinders which can be locked into place with the use of the user's hands or button/rotating switch. If a telescopic design is not used, it is envisaged a groove may be used to hold the speculum in place. There will be magnifying lens 13 for the user to see the ear canal.

FIGS. 3b (1) and (2) shows the add-on being a “flat” state to ensure it is portable. At each join, each section is locked so that the add-on maintains an ease of portability. It is envisaged that the product will be made of a light weight material. It is also envisaged that for the interests of sterility, this add-on may be disposable.

The following elements have not been shown but may be present in the device:

Within the shaft there may or will be a battery that may be disposable or rechargeable, to power the electrics of the invention/tool.

In the preferred embodiment, there will be a small circuit board, which control functions of the invention, which may or will be powered by the battery.

Light sources such as lamp or LED's, which will be used to power the ophthalmoscope and otoscope. There could be the use of a singular or multiple light sources for that. The light is preferably a white light that is not dangerous for short exposure to the human eye.

A storage device may be found in the shaft (e.g. hard disk) which may store any electronic media generated by the device.

Though this is the preferred embodiment that has been described in relation to FIGS. 1a to 1e, the invention is not restricted to these features by their number, variety or location. Should this add-on system of FIGS. 3a and 3b be used preferentially, then otoscope element in FIG. 1a-1e will become vacant for functionality. This may include a multitude of functions including not but exclusive to an electronic tuning forks, electronic stethoscope, Doppler probe, and thermometer.

The product may be made from a variety of material including but not exclusive to plastic and metals including aluminium. The aim of the material is to make the product light weight, sturdy and resistant to everyday wear and tear. These materials would be easily cleanable with health care setting cleaning solutions such as alcohol wipes.

FIG. 4a shows an embodiment of the fourth aspect of the invention. With the functional head 1 of an ophthalmoscope 40, there is a point of rotation 3 or a central axis 3 in the head that allows rotation on axis from a position inside the shaft 2 to a position outside of the shaft 2. There may be a locking mechanism in both the closed and open positions (not shown). This locking mechanism is under the user's control. The tool of this aspect of the invention may be made of strong material such as aluminium but not limited to that. The shaft 2 will have a cavity 45, shown in FIG. 4a′, that is moulded to the shape of the functional head to allow smooth fitting/reception of the head in the shaft.

FIG. 4b shows a side view of tool of this fourth aspect of the invention with a view of the axis 3 or point 3, which would allow rotational movement. The axis would be made from strong material such as aluminium but not limited to that. In FIG. 4b′ there is shown a protrusion 47 on the functional head 1 that may be provided to allow a user to hold onto this and rotate the head between the closed and open positions.

An alternate embodiment would be to have the functional head 1 on a hinge mechanism (not shown).

FIG. 5 shows an alternative embodiment of the fourth aspect of the invention, illustrated by reference to an ophthalmoscope 50. Here the functional head 1 is attached to the shaft 2 with a spring loaded mechanism (not shown), but is not limited to that particular mechanism. Again the shaft is provided with a cavity 55, shown in FIG. 5′, to receive the head. When the user wishes to not use the head 1, they can push down on the head so that head sinks into the shaft and can be locked inside the shaft. When the user wishes to use the tool, they may press down or press a button to release the head so it may automatically rise to a usable position. Alternatively a lever 56 may be provided, as shown in FIG. 5′, extending from the head 1 through the cavity 55 and out through a side wall of the shaft 2, to move the functional head between the in use and the protected positions. There is a single axis of movement which is in the same vector of the shaft position/along the longitudinal axis of the shaft. However there may be a possibility where that axis of movement is in a different plane of the invention.

FIG. 6 shows another embodiment of the fourth aspect of the device of the invention, again illustrated by reference to an ophthalmoscope 60. Here is there is a protective guard 4,that can move along the longitudinal axis of the shaft 2 to a position in which it will protect all or part of the functional head 1.

The guard 4 can be attached to the shaft 2 by a groove on each of the four sides of the shaft to receive runners provided in corresponding positions on inner surfaces of the guard 4 and can be locked into place.

The guard would be manufactured using light weight materials, which is strong and sturdy such as aluminium or plastic but not limited to these materials. The guard shown in FIG. 6 is rectangular in cross section having opposing open ends to allow the shaft 2 to be received and the guard to slide along the shaft.

The guard may preferably be a perfect fit to the shape of the functional head but is not limited to that.

FIG. 6′ shows a guard having a closed end 4a having an aperture 4b therein to receive the functional head 1 when the guard is slid into place to cover the functional head 1.

An alternative embodiment of this aspect of the invention (not shown) could be that instead of the guard sliding up and down the shaft, the guard may be fixed to the shaft with a hinge. This would provide an axis of movement for the guard, where in one position it would cover the functional head and in the other position be locked to the shaft so the functional head can be used by the clinician.

FIGS. 7a and 7b show a lens track for an ophthalmoscope that is designed to run over the viewing aperture. In FIG. 7a there is shown a lens track 70 having three lenses 71a, 71b, 71c mounted thereon. The lenses are spaced apart from each other along the length of the track.

In FIG. 7b there is shown an ophthalmoscope head 72 having a viewing aperture 73. The lens track 70 is positioned within the head 72 such that it runs over the viewing aperture 73 and in a plane parallel or coextensive with the longitudinal axis of the shaft 74 on which the head is mounted. The spacing of the lenses 71a, 71b, 71c is such that when one lens, for example 71a, is positioned in the viewing aperture the other two lenses, 71b, 71c, are not positioned to impinge on the viewing aperture. The lens track 70 in the areas between the lenses 71a, 71b, 71c is sized, shaped and made from a material that does not impact on the view of the user through the viewing aperture.

While the preferred embodiments have been described using specific terms, this is purely for illustrative purposes. It is to be understood that the descriptions are not limiting. It will be apparent to those of ordinary skill in the art that various modifications, substitutions, omission and changes may be made without departing from the spirit of the present invention. As such, it is intended they will fall within the scope of the present invention.

Another way of describing the first aspect of the invention is as follows:

The current invention is envisaged to have multiple axes of functions, to which two are demonstrated. This has been defined in this patent application as “functional axes”. It is envisaged that there will be at least two functional axes, which maybe of any angle apart in a 3 dimensional space. Each functional axis will have one or more functions and there are a limitless number of combinations in both number and variety of functions there are on each axis. It is also envisaged that there may be a need to have an axis where no function has been designated.

In its preferred embodiment, there will be two functional axes that are perpendicular to each other. In one functional axis, the two ends of the invention may comprise of an ophthalmoscope, tuning fork or otoscope. It is envisaged that the user of the tool may hold the opposing arm and pivot to use the function. The arms of the tool may be ergonomically designed to maximise user comfort. It is envisaged the dexterity of the user is not relevant and may be used with either the right or left hand. If a tuning fork element is at one end, it is preferable that the user will hold the arm with the tuning fork to stabilise that element on the patient's body.

In its second axis, the arms can come together and by doing so will form a handle. It can be seen that the second axis is at 90 degrees to the first axis. To use the tendon hammer aspect, the user will pull out the head of the tendon hammer and extend a telescopic shaft. The head of the hammer when the tool is its closed position will be in the same plane as the tool and will fit into the body of the tool. There will be an arcuate embedment in the two arms to allow the tendon hammer head to be fitted in.

In its preferred function, one axis is accessible per open state.

The tendon hammer shaft will be telescopic in nature will be comprised of shafts that when in use can be extended and fixed into shape. The tendon hammer head will consist of a central disc and a rubber ring surrounding the central disc. When the tendon hammer is to be used, the shaft will be extended and tendon hammer head can be manoeuvred so that it is now perpendicular to the shaft and be locked into position. The user would then hold the two arms of the tool and use the tendon hammer to test patients' reflexes. It is proposed that the telescopic shaft and central ring can be constructed of a lightweight metal and that there is a rubber ring surrounding the head.

The drawings can also be described alternatively as follows, in the following description, like reference characters designate like corresponding parts in its several views. Terms to describe position such as left/right/upper/low are used for convenience and to demonstrate the invention. They are not used to limit the invention's scope.

It is envisaged that the tool will have a multitude of functions including a retractable tendon hammer, an ophthalmoscope, an otoscope, red hat pin, visual display, camera functions, input/output devices. The preferred embodiment will be approximately less than 15 cm in length, less than 3-4 cm in depth and light weight.

FIG. 1A shows the product in its closed form. The ophthalmoscope (1) and otoscope (2) are two heads with the relevant optics in the shaft or “arms” of the device. Please note discrepancy in the widths of the two tool heads, where the wider head also contains the telescopic shafts of the reflex hammer (3). It is envisaged that this is the preferred embodiment. An arrangement may be possible where the tendon hammer shaft may be outside both of the heads of the instrument. (4) Represents a focal point of the instrument for the vibration.

FIG. 1B represents a side view of the instrument. There is a hinge behind the tendon hammers head, connects the two arms of the tool, and allows arc like motion of the two arms, to an angle to at least 180 degrees. Having the arms move to 180 degrees allows the two arms to be in the same plane and eases holding the tool for the use with the ophthalmoscope and other elements. The tendon hammer would not be connected to the hinge.

In reference to 1C, this diagram demonstrates one functional axis of the instrument with the use of the tendon hammer. It is preferred a Queens Square hammer is to be used, but the invention is not limited by that. When the user wishes to use the tendon hammer, they will pull out the head to reveal a series of telescopic shafts that will lock when fully extended. The user will then use the two arms of the tool as a handle to be able to swing the tendon hammer. The telescopic shafts can be made from plastic or a light weight metal such as aluminium. The head of the tendon hammer can then be made perpendicular to the shaft and be used to elicit tendon reflexes. When the tendon hammer is no longer to be used, the head is returned to its neutral position and returned to the closed position shown in FIG. 1A. As written earlier, it envisaged that than an arcuate shape is cut into the arms to allow the head of the tendon hammer to fit neatly into the tool. It is envisaged that invention is designed to fit ergonomically in the hand when using the tendon hammer. In its closed form it is envisaged that the device will have a master off key (not shown), which will automatically switch off all functions.

FIG. 1D reveals the second functional axis, when the two functional ends are being used. It can be seen that these two axes are ideally perpendicular to each other. The arrow denotes the range of motion needed to open up the device from its closed form. When the device is in its open form, it may become locked to prevent accidental closure. One end is the ophthalmoscope and the other end will be the otoscope. On the ophthalmoscope end, there is envisaged for a button to switch on the light source. There may be a separate button to vary the intensity of the light so that it does no blind the patient. There will also be a button/wheel, but not limited to that, to allow the lens of the ophthalmoscope to be changed. It is also possible that a lens free ophthalmoscope may be incorporated as a way to reduce costs and complexity of the tool. (7) represents the otoscope end, which will be discussed further on. Not shown is that device may include cameras at each end to take images of the eye or ear canal. These images will be stored preferably digitally onto a storage device within the tool. To use the ophthalmoscope, the user would hold the opposing arm of the invention and use the tendon hammer head as stability. Though the tool's two arms are preferably at 180 degrees, it may be preferable that the two arms are at a more acute angle to match the contours of a patient's face and allow the ophthalmoscope closer access to the eye.

The tuning fork element of this tool is incorporated as followed in the preferred embodiment. There would be a battery operated vibration generator (e.g. piezoelectric device) which is either sited at the tip of the tool (4) or within the shaft with a means of conducting the transmission. The tip of the tool is chosen because it can provide a precise point of vibration in test of the neurological vibration examination. The vibration would be isolated in the tip so that the whole tool does not vibrate when in use. There could be buttons or a dial on the side allowing the user to choose the frequency to vibrate including an on/off switch.

FIG. 1E shows the external features of the device. This can include a screen, which will have a multitude of functions including but not limited to; touch screen allowing the user to choose functions of the tool, pictures taken by the cameras on the device. There will be buttons/switches to allow a selection of the frequencies but not limited to those on the diagram in terms of number (16). (14) and (15) represents input/output sockets for the tool including but not limited to headphones, output to hardware such as computers, an electrical charger point, USB port. A red hat pin may be stored via a locking mechanism in the tool and be easily obtainable (18). The device may contain sound recording abilities especially if a stethoscope is to be incorporated in its design, and controls would available for playback, recording, rewind and fast forward (19). (20) represents a microphone that may be used to record sounds. It is purely for illustrative purposes that these functions are found on one arm of the invention and not indicative of the preferred embodiment.

FIG. 2 demonstrate the otoscope head. In its useable state (FIGS. 2A and 2B) there is a magnifying lens (6) and protrusion component (7) where the ear speculum sits on. The protrusion in this device will made of very small telescopic parts, which interlock when they are extended. This may be controlled by a switch or button, either mechanically or electrically (8).

FIG. 2C shows this device when telescopic parts are wound up to a closed position. It can be seen that such a configuration allows the otoscope now to be uniform and streamline and benefits portability.

To use the otoscope, the user put the tool so that the two arms are in a 180 degree angle and then will hold onto tool at the opposing arm and again uses the tendon hammer head as a grip. It is envisaged that there could be a button on the side that arm that allows the light to be turned on and off. There may be a separate dial to vary the intensity of the tool and that circuitry would be in the shaft of the tool.

FIGS. 3A and B show the add-on part, which may be used to convert an ophthalmoscope into an otoscope. In FIG. 3A, the add-on is represented by (9) and in this representation divided into three sections. No limit is placed on the number of sections needed for the device. At each join at each section (11) it is envisaged that joint is flexible but can be locked. The add-on can be held into position with a locking mechanism at the ophthalmoscope head and add-on (10). The ear speculum is held into place with a protrusion part, which may be made of telescopic cylinders which can be locked into place with the use of the user's hands or button/rotating switch (12).

If a telescopic design is not used, it is envisaged a groove may be used to hold the speculum in place. There will be magnifying lens for the user to see the ear canal (13).

FIG. 3B shows the add-on being a “flat” state to ensure it is portable. At each join, each section is locked so that the add-on maintains an ease of portability. It is envisaged that the product will be made of a light weight material such as the ear. It is also envisaged that for the interests of sterility, this add-on may be disposable.

1. A device comprising a shaft having at least a first portion and a second portion that can adopt a closed position in which the first and second portions are positioned adjacent each other and an open position in which the first and second portions are positioned distant from each other of the first or second portions housing or forming part of at least one device selected from the group an ophthalmoscope, an otoscope, a tendon hammer, a tuning fork and a red hat pin.

2. The device according to claim 1 wherein the first and second portions of the shaft are accurate in cross section and elongate.

3. The device of according to claim 1 wherein the first and second portions each have an outer surface that, when in the closed position, forms the outer surface of the shaft that faces the user, and an inner surface which when in the closed position faces into the centre of the shaft.

4. The device according to claim 1 wherein the first and second portions of the shaft each have a first end and a second end and are secured together by means of a pivotal connection between the second ends.

5. The device according to claim 1 wherein one of the first and second portions is provided with an ophthalmoscope head secured thereto or integrated therein.

6. The device according to claim 1 wherein one of the first and second portions is provided with an otoscope head secured thereto or integrated therein.

7. The device according to claim 1 wherein an ophthalmoscope head is secured to or integrated in one of the first and second portions and an otoscope head is secured to or integrated in the other of the first and second portions.

8. The device according to claim 1 wherein one of the first and second portions of the shaft is provided with the handle of a tendon hammer secured thereto.

9. The device according to claim 8 wherein the portion of the shaft having the handle of the tendon hammer secured thereto is provided with an aperture to receive and expose the head of the tendon hammer.

10. The device according to claim 9 wherein the handle of the tendon hammer is telescopic to allow the head to be extended clear of the shaft and the tool in the closed position to be used as the handle for the shaft.

11. The device according to claim 1 wherein there is provided a tuning fork.

12. The device according to claim 11 where vibration is provided and is focal and can be located anywhere on the device.

13. The device according to claim 11 wherein there is provided a speaker function to replicate a mechanical tone and tone decay.

14. A device according to wherein a red hat pin is releasably secured to one of the first and second shaft portions.

15. An otoscope head having a light source and magnifying lens and a protruding element for attachment of an ear speculum, wherein the protruding element is telescopic in nature and can be extended for use and retracted for storage.

16. The otoscope head according to claim 15 wherein the protruding part comprises cylindrical shafts, which can be extended out or remain in a closed position.

17. A device comprising at least one of a magnifying lens, provided with means to secure it to an ophthalmoscope, or a protruding element for attachment of an ear speculum, provided with means to secure it to an ophthalmoscope.

18. The device according to claim 17 wherein the device comprises both a magnifying lens and a protruding element for attachment of an ear speculum and is provided with one or more means to secure it to an ophthalmoscope.

19. The device according to claim 18 wherein the protruding element is telescopic in nature such that it can be extended for use and retracted for storage.

20. The device according to claim 18 wherein the device comprises three sections and each section is attached to an adjacent section by a link.

21. A medical tool having a head and a shaft wherein the shaft is provided with a suitable shaped, sized and positioned cavity and the head is mounted on the shaft by such means that the head can adopt an open position in which it is exposed ready for use and a closed position in which it is housed within the cavity in the shaft for protection.

22. The medical tool according to claim 21 wherein the head is secured to the shaft by means of a hinge or by means of a point of rotation or pivot.

23. The medical tool according to claim 21 wherein the head is secured within the shaft by a resilient material or device that favours pushing the head into the open position but will allow receipt of the head within the cavity.

24. The medical tool according to claim 23 wherein the resilient material or device is a coil spring.

25. A medical tool having a shaft and a head, wherein the shaft is provided with a guard movably secured thereto that in an open position exposes the head for use and in a closed position covers all or part of the head for protection.

26. The medical tool according to claim 25 wherein the guard is secured to the shaft by means of one or more hinges.

27. The medical tool according to claim 25 wherein the guard is slidingly moveable in relation to the shaft.

28. The medical tool according to claim 26 wherein the guard is secured to the shaft by a runner and track/groove arrangement.

29. The medical tool according to claim 28 wherein the or each groove is provided on the outer surface of the shaft or the inner surface of the guard and the or each corresponding runner is provided on the inner surface of the guard or the outer surface of the shaft respectively.

30. An ophthalmoscope having a viewing aperture and at least one lens held in a lens track, wherein the lens track runs over the top of the viewing aperture.

31. The ophthalmoscope according to claim 30 wherein the lens track runs in a plane parallel to or co-extensive with a longitudinal axis of a shaft of the ophthalmoscope.

32. The ophthalmoscope according to claim 30 wherein the lens track is supported by two or more wheels, around which the track passes.

33. The ophthalmoscope according to claim 30 wherein the lens track is arranged so that only one lens is visible through the viewing aperture at any one time.
